(19) United States
(12) Patent Application Publication
(10) Pub. No.: US 2022/0130541 A1
(43) Pub. Date: Apr. 28, 2022

(54) DISEASE-GENE PRIORITIZATION METHOD AND SYSTEM

(71) Applicant: KING ABDULLAH UNIVERSITY OF SCIENCE AND TECHNOLOGY, Thuwal (SA)

(72) Inventors: Xin GAO, Thuwal (SA); Yu LI, Thuwal (SA); Hiroyuki KUWAHARA, Thuwal (SA)

(21) Appl. No.: 17/422,547

(22) PCT Filed: Jan. 27, 2020
(86) PCT No.: PCT/IB2020/050614

§ 371 Date: Jul. 13, 2021

Related U.S. Application Data
(60) Provisional application No. 62/808,581, filed on Feb. 21, 2019.

(51) Int. Cl. G16H 50/20; G16H 50/70; G16H 70/60; G06N 3/04; G06N 3/08; G16B 40/00; G16B 5/20

(52) U.S. Cl. CPC G16H 50/20; G16H 50/70; G16H 70/60; G16B 5/20; G06N 3/08; G16B 40/00; G06N 3/04

(57) ABSTRACT

A method for disease-gene prioritization includes building a heterogenous network to include gene nodes gj and disease nodes di; supplying additional information (x_{di}, x_{gj}) related to the gene nodes gj and the disease nodes di to generate embeddings zₖ associated with the gene nodes gj and the disease nodes di; applying a graph convolutional neural network model G to the heterogenous network and to the embeddings zₖ to calculate aggregated embeddings zₖ₊₁; and estimating, with an edge decoder model ED, a probability P of an edge (di, gj), between a selected gene node gj and a selected disease node di. The edge (di, gj) between the selected gene node gj and the selected disease node di is the disease-gene prioritization.

### CROSS-REFERENCE TO RELATED APPLICATIONS

This application claims priority to U.S. Provisional Patent Application No. 62/808,581, filed on Feb. 21, 2019, entitled “DEEP LEARNING-BASED DISEASE-GENE PRIORITIZATION METHOD,” the disclosure of which is incorporated herein by reference in its entirety.

### BACKGROUND

### Technical Field

Embodiments of the subject matter disclosed herein generally relate to a system and method for prioritization of candidate genes to the genome-based diagnostics of a range of genetic diseases and more particularly, using a novel graph convolutional network-based disease-gene prioritization method, PGCN, through the systematic embedding of a heterogeneous network made by genes and diseases, as well as their individual features.

### Discussion of the Background

The last decade has seen a rapid increase in the adoption of whole-exome sequencing in the clinical diagnosis of genetic diseases. However, the success rate of such genome-based diagnostics still remains far from perfect, with reported yields for a range of Mendelian diseases ranging from ˜20 to ˜50%. This relatively low-yield is largely attributed to a considerable difficulty in differentiating disease-causing variants from a large pool of rare genetic variants that are not pathogenic and do not play roles in the expression of the disease phenotype.

To efficiently detect pathogenic variants and to improve the diagnostic rate of the genome-based approach, it is necessary to have disease-gene prioritization that substantially reduces the number of candidate causal variants and ranks them for further interrogations based on the association of the corresponding genes with the disease phenotype. In other words, the disease-gene prioritization is the process of assigning a likelihood of gene involvement in generating a disease phenotype.

A number of computational methods have been developed to tackle the disease-gene prioritization problem and have been shown to be useful. For example, Endeavour was able to associate GATA4 with congenital diaphragmatic hernia; GeneDistiller discovered the role of MED17 mutations in infantile cerebral and cerebellar atrophy. Based on the underlying computational techniques, existing disease-gene prioritization methods can be categorized into five types.

The first type is the filter methods, which sift the candidate list of genes into a smaller one according to the properties that associated genes should have. The second type of methods is based on text mining. Such methods score the candidate genes using the co-occurrence evidence with a certain disease from the literature. Thus, these methods can only detect associations that are already known. The third type is similarity profiling and data fusion methods. This is the dominant type in the disease gene prioritization community and includes the famous Endeavour method. These methods are based on the idea that similar genes should be associated with similar sets of diseases and vice versa. The similarity measurement can be defined using different data sources, such as Gene Ontology (GO) or the BLAST score. After obtaining the similarity scores from each data source, such methods apply data fusion to aggregate these scores into a global ranking. The fourth type is network-based methods, which are discussed in [1] to [8]. Such methods represent diseases and genes as nodes in a heterogeneous network, in which the edge weight represents their similarities. The last type is based on matrix completion techniques in recommender systems. These methods represent the disease-gene association as an incomplete matrix and solve the disease-gene prioritization problem by filling the missing values of the matrix. This category of methods has been shown to be the state-of-the-art at present.

Despite the advances of the existing methods, they have the following problems. Firstly, the similarity-based methods, which are rooted in the “guilt-by-association” principle, often fail to handle new diseases whose associated genes are completely unknown. Secondly, although the performance of the network-based methods is reasonable, they are biased by the network topology and cannot easily integrate multiple sources of information about genes and diseases. Thirdly, the matrix completion methods assume and look for a weighted linear relationship between genes and diseases, which, in reality, is most likely to be highly nonlinear. In addition, most of the existing methods rely heavily on manually-crafted features or pre-defined rules of data fusion.

Therefore, the disease-gene prioritization problem remains elusive. On the other hand, the recent success of graphical models and deep learning in bioinformatics [10] to [14] suggests the possibility to systematically incorporate multiple sources of information in the heterogeneous network and learn the highly nonlinear relationship between diseases and genes.

Thus, there is a need for a new method and system that prioritizes the disease-gene link and avoids the problems mentioned above.

### BRIEF SUMMARY OF THE INVENTION

According to an embodiment, there is a method for disease-gene prioritization, and the method includes building a heterogenous network to include gene nodes gj and disease nodes di; supplying additional information (x_{di}, x_{gj}) related to the gene nodes gj and the disease nodes di to generate embeddings zₖ associated with the gene nodes gj and the disease nodes di; applying a graph convolutional neural network model G to the heterogenous network and to the embeddings zₖ to calculate aggregated embeddings zₖ₊₁; and estimating, with an edge decoder model ED, a probability P of an edge (di, gj), between a selected gene node gj and a selected disease node di. The edge (di, gj) between the selected gene node gj and the selected disease node di is the disease-gene prioritization.

According to another embodiment, there is a computing device for producing a disease-gene prioritization, and the device includes an input/output interface for receiving additional information (x_{di}, x_{gj}) related to gene nodes gj and disease nodes di to generate embeddings zₖ associated with the gene nodes gj and the disease nodes di; and a processor connected to the input/output interface and configured to, build a heterogenous network made by the gene nodes gj and the disease nodes di; apply a graph convolutional neural network model G to the heterogenous network and the embeddings zₖ to calculate aggregated embeddings zₖ₊₁; and estimate, with an edge decoder model ED, a probability P of an edge (di, gj), between a selected gene node gj and a selected disease node di. The edge (di, gj) between the selected gene node gj and the selected disease node di is the disease-gene prioritization.

According to still another embodiment, there is a method for training a graph convolutional neural network model G for disease-gene prioritization. The method includes building a heterogenous network from gene nodes gj and disease nodes di; supplying additional information (x_{di}, x_{gj}) related to the gene nodes gj and the disease nodes di to generate embeddings zₖ associated with the gene nodes gj and the disease nodes di; applying the graph convolutional neural network model G to the heterogenous network and the embeddings zₖ to calculate aggregated embeddings zₖ₊₁; estimating, with an edge decoder model ED, a probability P of an edge (di, gj), between a selected gene node gj and a selected disease node di; and repeating the above steps until the probability P is one for a known connection between the selected gene node gj and the selected disease node di. The edge (di, gj) between the selected gene node gj and the selected disease node di is the disease-gene prioritization.

### BRIEF DESCRIPTION OF THE DRAWINGS

For a more complete understanding of the present invention, reference is now made to the following descriptions taken in conjunction with the accompanying drawings, in which:

FIG. 1 illustrates a heterogenous network that describes genes, diseases, and links between genes and diseases;

FIGS. 2A and 2B illustrate additional information that is added to the heterogeneous network;

FIG. 3 schematically illustrates how the additional information is propagated through the network;

FIG. 4 schematically illustrates how a probability is calculated for each edge of the network;

FIG. 5 schematically illustrates how the probability is improved using a neural network system;

FIG. 6 is a flowchart of a method for calculating disease-gene prioritization;

FIG. 7 illustrates the overall performance of the novel method and five traditional methods;

FIGS. 8A to 8C further illustrate the performance of the novel method and the five traditional methods for different criteria;

FIGS. 9A to 9C illustrate the performance of the novel method and the five traditional methods for different tests; and

FIG. 10 schematically illustrates a computing device that can be used to implement any of the methods discussed herein.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a system and method that casts the disease-gene prioritization problem as a link prediction problem.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel disease-gene prioritization method, called herein “PGCN,” is developed based on graph convolutional neural networks (GCN) introduced by [10] and [15]-[17].Starting from a heterogeneous network, which is composed of a genetic interaction network, a human disease similarity network, and a known disease-gene association network, to which additional information about genes and diseases from multiple sources is added, the novel method first learns embeddings for genes and diseases through graph convolutional neural networks, by considering both the network topology and the additional information of diseases and genes. Such embeddings are fed into an edge decoding (edge prediction) model to make predictions for disease-gene associations. Although this method is described in two steps, the model used by the method is trained in an end-to-end manner so that the model can jointly learn the embedding and the decoding.

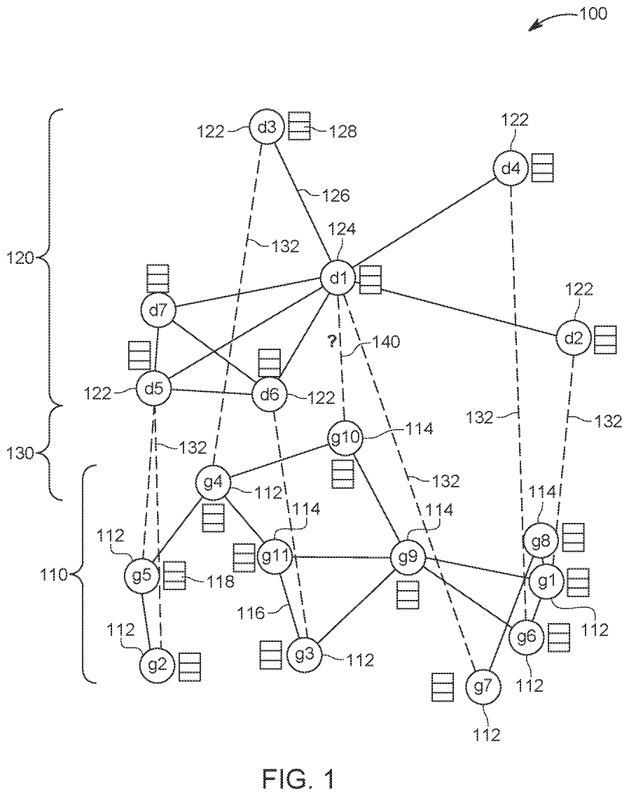

In one embodiment, the disease-gene prioritization problem is treated as a link prediction problem. Unlike previous studies which solve the problem with matrix factorization, the novel method uses graph convolutional neural networks. The method compiles the disease similarities, genetic interactions, and disease-gene associations into a multi-nodal heterogeneous network 100, as shown in FIG. 1. FIG. 1 shows that the multi-nodal heterogeneous network 100 includes a gene network 110, a disease network 120, and a gene-disease network 130. The gene network 110 includes genes 112 that are known to be associated with various diseases 122 from the disease network 120, and also includes genes 114 that are not currently associated with other diseases. The disease network 120 also includes diseases 124 that are not associated with any gene 112 or 114. The links 132 between the genes 112 and the diseases 122 form the gene-disease network 130. Note that each gene 112 or 114 has neighbor links 116 which indicate some gene interactions, while the diseases 122 and 124 have their own neighbor links 126, which indicate some similarity between the diseases. Each gene 112 or 114 has an embedding 118, which is discussed later, and each disease 122 or 124 has its own embedding 128, which is also discussed later. The algorithm to be discussed next is designed to find new gene-disease links 140. Because of the various and different networks 110, 120, and 130 involved in this method, the overall network 100 is considered to be a heterogenous network.

In this heterogenous network 100, the potential disease-gene associations or links 140 can be considered as missing links and the goal of this method is to predict (calculate a probability) these links. Thus, according to one embodiment, the method to be discussed next learns the nodes' latent representations (embeddings 118 and 128) from their initial raw representations (information encoded from different sources), considering the graph's topological structure and the nodes' neighborhood, after which the method makes predictions using the learned embeddings using the edge decoding model. Both the embedding model and the decoding model (which are discussed later) are trained in an end-to-end manner so that each model is optimized while being regularized by the other one. The components of the proposed method are discussed now in more detail.

Recent studies have formulated the disease-gene prioritization problem as a matrix completion problem and applied the recently developed methods in recommender systems, resulting in better performance than the previous state-of-the-art methods. Although the method proposed herein also considers the problem as a recommender system problem, the novel method treats the entire data structure as a heterogeneous network 100 as shown in FIG. 1. Each node 112, 114, 122, or 124 represents a disease or a gene, and each edge 132 represents one specific kind of interaction between a specific gene and a specific disease. In addition, each disease and/or gene is supplemented with additional information from different data sources, as discussed later. The goal of the method is to predict the potential links 140 between disease nodes and gene nodes, whose link strength can be used for prioritization. Compared to the matrix factorization methods, this formulation can capture the nonlinear relationship between the diseases and the genes. Compared to the traditional network-based methods, this novel method is able to integrate the information from different sources in a systematic and natural way.

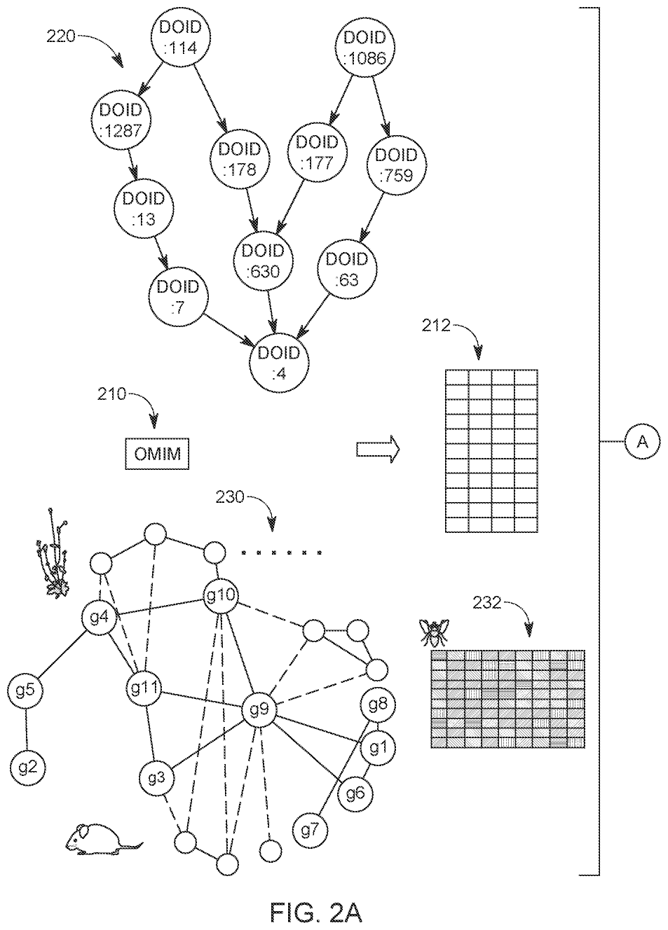

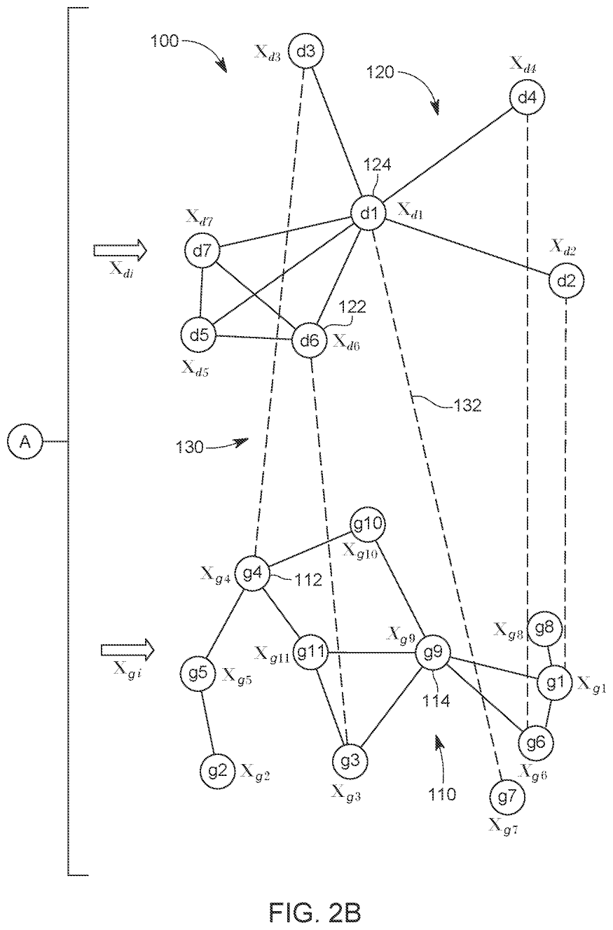

One component of the novel method is the graph convolutional encoder, which can learn the embeddings 118 and 128 from the nodes' neighborhood, node-specific information, and the topology of the heterogeneous network 100. A problem for learning the embeddings 118 and 218 from the graph data is to propagate and transform the associated information along the network 100. As shown in FIG. 2A, the entire graph starts from the heterogeneous network 100, with each node 112, 114, 122, or 124 containing information from different sources. In the graph convolution model G, each node's neighboring nodes defines the computational graph of its local neural network, i.e., its own neural network architecture. Although the local computational graphs can be different for different nodes, the same operations share the same parameters and activation functions, which specify how the information is shared and propagated across the computational graph.

Because the method instantiates the graph convolution operation using a fully-connected neural network, the model G can seamlessly integrate information from different sources. The embeddings are fed into the link decoding model as discussed later. Thus, the proposed method can achieve problem-specific data integration systematically, whose parameters are learned from the data in an end-to-end manner.

As previously discussed, the network 100 in the model of FIG. 1 is a heterogeneous network containing three components: the gene network 110, the disease similarity network 120, and the disease-gene network 130. The disease-gene network 130 may be built from the Online Mendelian Inheritance in Man (OMIM) database 210, which is schematically illustrated in FIGS. 2A and 2B and which is an online Catalog of Human Genes and Genetic Disorders (Nov. 26, 2017), with the associations being the links. After preprocessing, this network contains 12,331 genes, 3,215 diseases, and 3,988 disease-gene associations.

For the gene network 110, the method used the HumanNet database. This large-scale functional gene network was constructed by considering multiple sources of information, including human mRNA co-expression, protein-protein interactions, protein complex, and comparative genomics information. In total, it incorporated 21 genomics and proteomics datasets from four species. Compared to the network built from the single dataset, such as protein-protein interaction networks, it has higher accuracy and genome coverage. The usefulness of the HumanNet in the disease gene prioritization has been proved by previous studies. In summary, the gene network 110 is composed of 12,331 genes and 733,836 edges with positive weights. Those skilled in the art will understand that more or less information can be used for any of the three networks 110, 120, and 130.

The disease similarity network 120 used the MimMiner network. This network was built by using text mining analysis on the OMIM database 210. For each disease, the anatomy and disease sections of the medical subject headings were used to extract terms from the OMIM database 210, whose frequencies were used as the feature vectors of the disease. After further refinement, the feature vectors were used to compute the pairwise similarities between the disease, which resulted in the MimMiner network. Although in the construction process it did not involve gene information, the similarities were shown to be positively correlated with a number of measures of gene function. This network has also been used as a feature input in the previous disease-gene prioritization methods [8].After setting the similarity threshold as 0.2, a disease similarity network with 3,215 diseases and 645,945 edges was obtained.

In contrast to the existing network-based methods, the model 100 can naturally incorporate additional information about the nodes from different sources, i.e., the novel method is generic and can take any source of information for diseases and genes. In one implementation, the model 100 incorporated, as illustrated in FIGS. 2A and 2B, two kinds of additional information for the disease nodes. The first data source is the Disease Ontology (DO) similarity 220. After collecting the ontology for the disease nodes, a similarity matrix was calculated for those diseases using the Resnik pairwise similarity with the best-match average (BMA) strategy. For each disease, the method took the corresponding row of this matrix as an additional feature vector for this node.

The second data source is the clinical text from the OMIM webpages. The Clinical Feature and Clinical Management sections were collected from the OMIM webpages for each disease, and the most frequent and most rare words were removed. Then, the frequency of each unique word in the corpus related to each disease was counted. To remove the bias of the relatively frequent words, the method applied the TF-IDF scheme 212 to the term frequency matrix and obtained the corresponding row as the feature vector x_{di} for a disease. Finally, the two vectors were concatenated as the additional information for the disease.

The method also used two kinds of features as the additional information for the gene nodes of the gene network 110. The method collected the microarray measurement of the gene expression level in different tissue samples from BioGPS and Connectivity Map. Since some genes are missing in the probes, the method obtained 4,536 features for 8,755 genes. It is well-known that samples from the same cell type of different individuals tend to have a similar expression pattern, which results in redundant information in the obtained feature matrix. To eliminate the redundancy and reduce the dimensionality, the method applied the principle component analysis (PCA) on the features and used the first 100 eigenvectors as the feature representations from gene expression microarray.

The second type of additional information for genes is derived from the gene-phenotype associations 230 of other species. Following the previous studies [8], the method used the phenotypes from eight species. As a result, the method obtained eight matrices, whose rows represent different genes and the columns represent the phenotypes of different species. The method concatenated those gene-phenotype matrices together with the microarray matrix 232 along the gene dimension, resulting in the additional information x_{gi} of the genes. The additional information x_{di} and x_{gi} was added to each corresponding node in the disease network and the gene network, respectively, as schematically illustrated in FIGS. 2A and 2B.

Based on this additional information x_{di} and x_{gi}, the embeddings 118 and 128 are now constructed using graph convolutional neutral networks, by taking into account the network topology, the nodes' neighborhood, and the additional information associated with each node. Formally, the embeddings are constructed by considering a graph [symbol] =(V, ε), where V represents the set of nodes and ε represents the set of edges, with the adjacent matrix being A. The additional information of a node i ϵ V is denoted as xᵢ ϵ [symbol] ^{m}^{ᵢ}. Note that in this embodiment, the value of mᵢ, which represents the dimension of the additional feature vectors, can be different for different kinds of nodes, i.e., gene nodes and disease nodes. The goal of embedding is to map each node i to an embedding vector zᵢ ϵ [symbol] ^{c}, where c<<mᵢ, considering the information contained in A and {xᵢ}ᵢ₌₁^{|v|}.

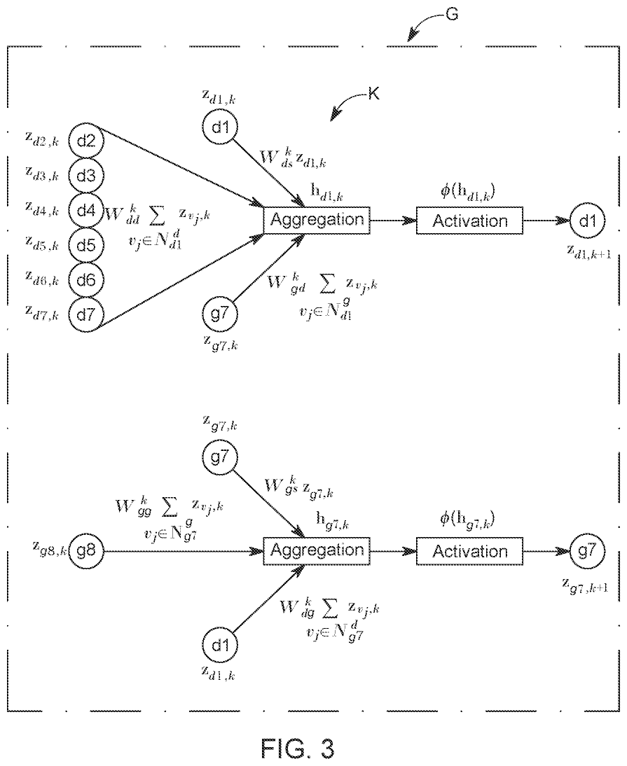

A problem of learning the embeddings (or embedding vector z) with the graph convolutional neural network is to figure out how to transform and propagate information (the additional information and intermediate embeddings of each node) across the entire network. In this embodiment, the GCN module defines the information propagation architecture (the local computational graph) for each node using the node's neighborhood in the graph corresponding to the network 100. Note that FIG. 3 shows a single layer of the model G. In terms of the parameterization of the local computational graph, which defines how the information is propagated and shared in the model G, the parameters and weights are shared across all the local computational graphs built from graph of the network 100, with the assumption that within the same graph representing the network 100, the way of sharing and propagating information should be the same. As a result, for a given node i, each layer of the graph convolutional neural network model G aggregates and transforms the information (feature representations) from its neighbors and applies the same transformation to all parts of the network.

In this regard, FIG. 3 shows how the information from the disease nodes d1 to d7 and the gene node g7 is aggregated to generate the aggregated embedding z_{i,k} of the disease node d1. FIG. 3 also shows how the information from the gene nodes g7 and g8 and the information from the disease node d1 is aggregated to obtain the aggregated embedding of the gene node g7. The neighboring nodes are selected based on the links illustrated in the network 100. Also note that each node for which the aggregated embedding is calculated is also represented with a given weight.

If there is only one layer of the graph convolution model G, as illustrated in FIG. 3, the embedding will only aggregate information from its first-order neighbors. Thus, stacking N layers of the graph convolutional model G′s layers can make the embedding effectively convolve information from its N-order neighbors explicitly. In another embodiment, when more than one graph convolutional layer is stacked, the information of each single node can start broadcasting to the entire network implicitly, whose effect depends on the network topological structure (size, connectivity etc.). By using multiple convolutional layers, it is possible to learn the embedding of nodes, considering the network topology, local neighborhoods, and additional information of the nodes.

Formally, in each layer k of the model G, for each node i, the information aggregation and transformation model h_{i,k} illustrated in FIG. 3 is given as follows:

$$\begin{matrix}{h}_{i , k} = {\sum }_{l} {\sum }_{j ∈ {𝒩}_{i}^{l}} ( {c}_{i , j} {W}_{l}^{k} {z}_{j , k} + {W}_{{t}_{i} , s}^{k} {z}_{i , k} ) \\ with & ( 1 ) \\ {z}_{i , k + 1} = ϕ ( {h}_{i , k} ) & ( 2 )\end{matrix}$$

where z_{i,k} ϵ [symbol] ^{c}^{ₖ} is the aggregated embedding, or the hidden representation (note that a hidden representation is layer that is neither the input layer nor the output layer of the model G) of node i in the k-th graph convolutional layer, and cₖ is the dimensionality of that hidden representation; h_{i,k} represents the feature vector which has aggregated the information from the k-th layer hidden representations of the node's neighbors (see also FIG. 3); I represents the link type, i.e., genetic interaction, disease-disease similarity, or disease-gene association; [symbol] are the neighbors of node i, which are linked by the link type I; Wₗ^{k} is the weight parameter related to the link type I, such as W_{dg}^{k}, W_{gd}^{k}, W_{dd}^{k} and W_{gg}^{k}, as illustrated in FIG. 3; c_{i,j} is the normalization constant [10], which is defined as c_{i,j}=1/\sqrt{||||}; Wₜ_{ᵢ}_{,s}^{k} is the weight parameter preserving the information from the node itself, where tᵢ indicates the type of the node; and ϕ is a non-linear activation function, which is usually chosen as the rectified linear unit (ReLU). Note that the above aggregation and transformation formulas are related to all the neighbors of a certain node i, which means that the computational graph architecture can be different for nodes with different local neighborhood structure. FIG. 3 shows two examples of two very different computational graphs for nodes d1 and d7. Although the computational graphs can be different, the parameters are only related to the link type, not related to the node neighborhoods, which means that the parameterization is shared across the entire graph.

In this method, the summation is used as the information aggregation method in the GCN model. With different information aggregation methods, it can result in different GCN variants. However, no matter which method is chosen, the aggregation and transformation layer convert the hidden representation of node i in layer k, z_{i,k}, into the hidden representation in the next layer as Z_{i,k+1}. The output of the last graph convolutional layer, z_{i,N}, is used as the final embedding 118 or 128 for that node, zᵢ. With these selections, the input of the first convolutional layer is the original feature vector of each node, i.e., z_{i,0}=xᵢ.

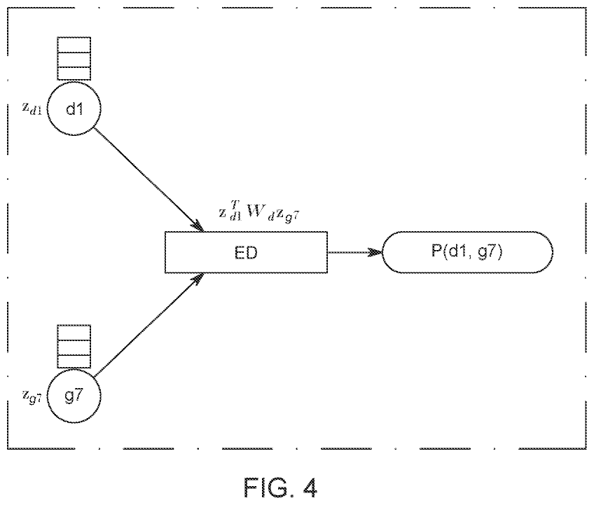

Having described how to construct the embedding 118 or 128 of each node in FIG. 1, based on the model G shown in FIG. 3, and equations (1) and (2), an edge decoder ED, which predicts or estimates a probability P associated with the edges for unliked nodes, based on the aggregated embeddings calculated above, is now discussed with regard to FIG. 4. A bilinear decoder ED is used as the edge decoder, and the decoder ED has, in one embodiment, the following mathematical form:

P(dᵢ,dⱼ)=σ(z_{d}_{ᵢ}^{T}W_{d}z_{g}_{ⱼ}), (3)

where z_{d}_{ᵢ}^{T} ϵ [symbol] ^{c} is the learned embedding of a disease node dᵢ; z_{g}_{ⱼ} ϵ [symbol] ^{c} is the learned embedding of a gene node gⱼ; W_{d} is the trainable parameter matrix, which models the interaction between each two dimensions of z_{d}_{ᵢ}^{T} and z_{g}_{ⱼ}; and σ is the sigmoid function, which converts the output value of the edge decoder to the range of (0, 1), as a probability value. In one embodiment, the sigmoid function is defined as

$$σ ( z ) = \frac{1}{1 - {e}^{- z}} .$$

The edge decoder ED is illustrated in FIG. 4 as having as input the learned embeddings of a disease node d1 and of a gene node g7 and as having as output the probability P of an edge defined by the disease node d1 and the gene node g7. Note that, similar to the graph convolutional neural network model G in FIG. 3, the parameters of the bilinear decoder model ED are also shared across different gene-disease pairs, which can effectively reduce the risk of overfitting.

Taking together the GCN model G illustrated in FIG. 3 and the edge decoder model ED illustrated in FIG. 4, the novel method has the following trainable parameters: (1) the link-type-specific and layer-specific convolutional weight parameters Wₗ^{k}, which suggest how to aggregate and transform information from the node's neighbors; (2) the node-type-specific and layer-specific weight parameters W_{t,s}^{k}, which indicate how to preserve and transform the nodes' self-information from one layer to the next; and (3) the weight parameters of the bilinear edge decoder model, W_{d}, which model the interaction between two dimensions of the input embeddings of two nodes. As shown in FIGS. 3 and 4, the GCN model G and the edge decoder model ED can be combined together to form an end-to-end model, which takes the raw representation of two nodes and output a final probability P_{f} between the two nodes, i.e., the probability P_{f} that there is a connection between the gene node and the disease node. Consequently, the entire model and all the parameters can be trained in an end-to-end manner.

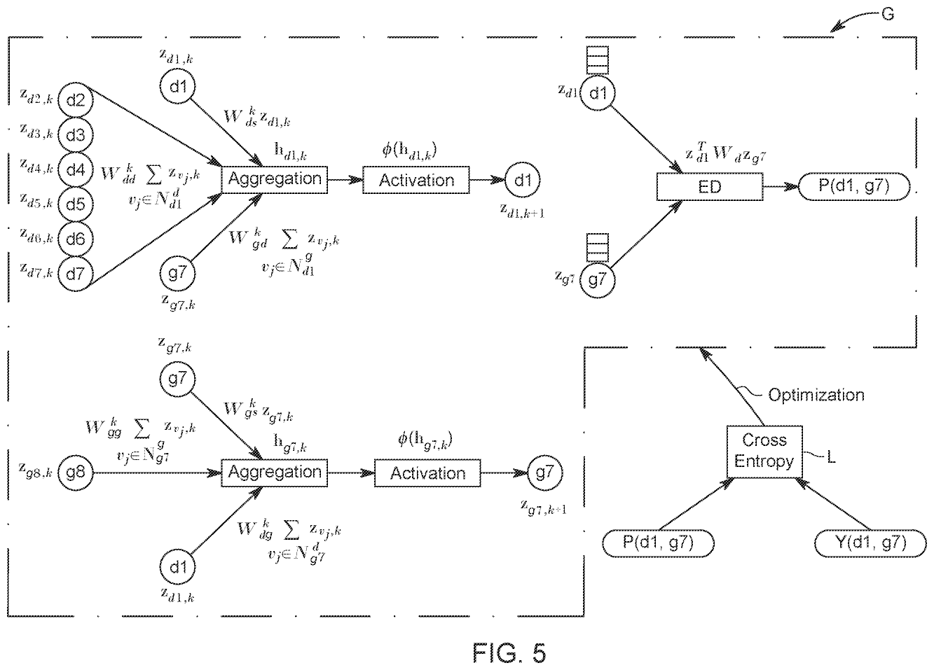

The hyper-parameters when building and training the model are now discussed. The cross-entropy loss L was used as the loss function to train the entire model G and ED, as schematically illustrated in FIG. 5. The cross-entropy loss L has the following form:

L(dᵢ, gⱼ)=−log P (dᵢ, gⱼ)− [symbol] log(1−P(dᵢ, gₙ)), (4)

where (dᵢ, gⱼ) defines an edge in the training data and [symbol] is an ensemble of loss related to a negative training set (that includes random linkages between two nodes). The second term is incorporated into equation (4) to force the model to recover the non-edges in the original graph. This means that the ground truth value Y(dᵢ, gⱼ)=1 in FIG. 5. Note that the initial probability P calculated with equation (3) is improved by applying the optimization problem illustrated by equation (4), so that the final probability P_{f} more accurately predicts the link between the gene node and the disease node under consideration. By using the cross-entropy loss L, it is desired that the model assigns the probabilities for the observed training edges as high as possible while assigning low probabilities for the random edges. Following the previous studies, this embodiment used negative sampling to achieve this goal, which is illustrated by the last term in equation (4), as previously discussed. For each existing edge (dᵢ, gⱼ), which is a positive sample, a random edge (dᵢ, gₙ) is sampled by randomly choosing the second node gₙ, which follows the sampling distribution P. Considering all the edges, the total cross-entropy loss of the model is given by:

$$L = {\sum }_{( {d}_{i} , {g}_{j} ) ∈ {ɛ}_{dg}} L ( {d}_{i} , {g}_{j} ) , \qquad ( 5 )$$

where ε_{dg} represents all the edges connecting the diseases and genes nodes shown in the network 100 in FIG. 1. As previously discussed, the model is trained in an end-to-end manner, where the loss function gradient is back-propagated to the parameters in both the CGN model and the edge decoding model ED. This end-to-end training strategy is more likely to find problem-specific, effective models and embeddings, which has been proved by previous studies.

In one embodiment, the above model has been implemented to have the number of layers 2, with the dimension of the hidden representation as 64 and the final embedding dimension as 32. The model was trained using an Adam optimizer, with the learning rate as 0.001. To reduce overfitting, this embodiment used the combination of dropout on the hidden layer unites with the dropout rate as 0.1, and the legendary weight decay method. The model's parameters were initialized using the Xavier initializer. During training, mini-batches of edges were fed to the model, with the batch size as 512. This can reduce the memory requirement and serve as an additional regularizer that further alleviates overfitting. In total, the model was trained for 300 epochs. With the help of a Titan Xp card, the training of the model was performed in 10 hours.

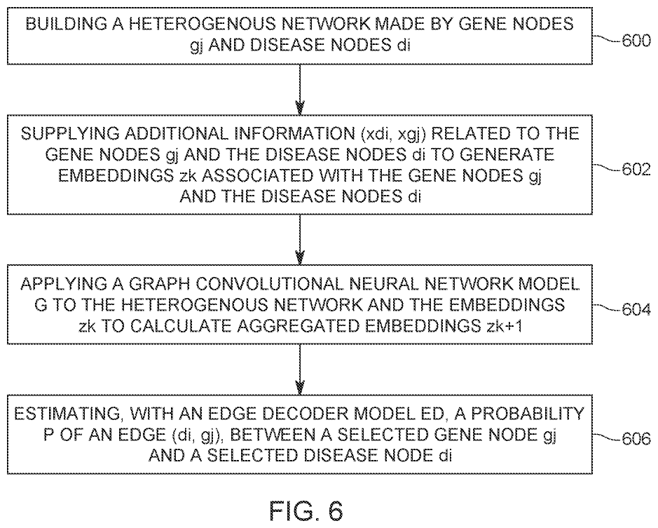

A method for disease-gene prioritization is now discussed with regard to FIG. 6. The method includes a step 600 of building a heterogenous network 100 made by gene nodes gj and disease nodes di; a step 602 of supplying additional information (x_{di}, x_{gj}) related to the gene nodes gj and the disease nodes di to generate embeddings zₖ associated with the gene nodes gj and the disease nodes di; a step 604 of applying a graph convolutional neural network model G to the heterogenous network 100 and the embeddings zₖ to calculate aggregated embeddings zₖ₊₁; and a step 606 of estimating, with an edge decoder model ED, a probability P of an edge (di, gj), between a selected gene node gj and a selected disease node di. The edge (di, gj) between the selected gene node gj and the selected disease node di is the disease-gene prioritization.

In one application, the step of applying a graph convolutional neural network model G includes aggregating, for the selected gene node, (1) embeddings z_{gk} of all gene nodes linked to the selected gene node, (2) an embedding z_{dk} of the selected gene node, and (3) embeddings z_{dk} of all disease nodes linked to the selected gene node to obtain a gene feature vector h_{dk}; and activating the gene feature vector h_{dk} with an activation function ϕ to obtain the aggregated embedding z_{g(k+1)} for the selected gene node. The step of applying a graph convolutional neural network model G may further include aggregating, for the selected disease node, (1) embeddings z_{dk} of all disease nodes linked to the selected disease node, (2) an embedding z_{dk} of the selected disease node, and (3) embeddings z_{dk} of all disease nodes linked to the selected disease node to obtain a disease feature vector h_{dk}; and activating the disease feature vector h_{dk} with an activation function ϕ to obtain the aggregated embedding z_{d(k+1)} for the selected disease node.

In another application, the step of aggregating, for a selected gene node or for a selected disease node, uses a different weight for each type of embedding. The method may also include training the graph convolutional neural network model G and the edge decoder model ED for each of the different weight. The step of estimating may include calculating the probability P as a sigmoid function applied to a product of (1) the aggregated embedding of the selected gene node, (2) a weight of the edge decoder model, and (3) the aggregated embedding of the selected disease node.

In one embodiment, the method may include applying a cross-entropy loss function L to the edge decoder model ED to calculate a final probability P_{f} of the edge (di, gj). The additional information includes one or more of an Online Mendelian Inheritance in Man, disease ontology, associations in other species, human mRNA co-expressions, protein-protein interactions, protein complex, comparative genomics interaction, and disease similarity network. The heterogenous network includes a gene network, a disease network, and a gene-disease network.

In one application, the step of building includes linking each gene node gj to other known gene nodes; linking each disease node di to other known disease nodes; and linking each gene node gj to the disease node di if such a link is known. The method may also include initializing the embeddings with the additional information. All the steps and features discussed above with regard to the method of FIG. 6 may be combined in any desired order.

To evaluate this novel method versus the traditional methods, the following criteria have been used: Area Under the Receiver Operating Characteristic curve (AUROC), Area Under the Precision-Recall Curve (AUPRC), Boltzmann-Enhanced Discrimination of ROC (BEDROC), Average Precision at K (AP@K), and Recall at K (R@K) score. AUROC is a commonly used criterion in machine learning, which computes the area under the ROC curve. In the disease-gene prioritization problem, it can be interpreted as the probability of a true disease-associated gene is ranked higher than a false one selected randomly in a uniform distribution. Similar to AUROC, AUPRC computes the area under the precision-recall curve. BEDROC, proposed to solve the “early recognition” problem, can be interpreted as the probability of a disease-associated gene being ranked higher than a gene selected randomly following a distribution in which top-ranked genes have a higher probability to be chosen. AP@K computes the precision of the prediction if one considers the top K predicted associations. Recall at K considers the recall score within the top K predictions. These five criteria can provide a comprehensive evaluation of the proposed novel method.

Prior to showing and comparing the results obtained with the novel method and the five traditional methods, the five competing methods are briefly introduced. The first method is Katz [8], which is a typical network-based method. It computes the node similarity based on the network topology. The similarity matrix is then used to make predictions for disease-gene associations. The second method is Catapult [8], another network-based method. It combines the supervised learning with social network analysis, and has been shown to be the state-of-the-art network-based method. This method deploys a biased support vector machine (SVM) as the classifier while the features are derived from random walks in the heterogeneous gene-trait network. This method significantly outperformed the previous network-based methods, such as PRINCE and RWRH. The third method is a recent network-based method, the Graph Convolution-based Association Scoring (GCAS) method [9].This method used the GCN as a pure network analysis tool which can perform information propagation on the similarity and association networks. The novel method discussed in FIG. 6 differs from the GCAS method in that the novel method uses the GCN model to integrate information from different sources and learn embeddings specifically for this problem, which are particularly suitable for the downstream edge prediction task. The fourth method is the Inductive Matrix Completion (IMC) method, which uses the matrix completion method into the disease-gene prioritization field for the first time. It constructs features from genes and diseases from multiple sources, ranging from gene expression array to disease similarity networks. It then learns low-rank latent vectors for diseases and genes, which can explain the observed disease-gene associations, taking into consideration features using a linear model. The learned latent vectors are then used for making further predictions. The last method is the very recently developed GeneHound method. It also utilizes the matrix completion method, but combines the Bayesian approach with the matrix completion, which takes the disease-specific and gene-specific information as the prior knowledge. This method has been shown to outperform the legendary Endeavour method.

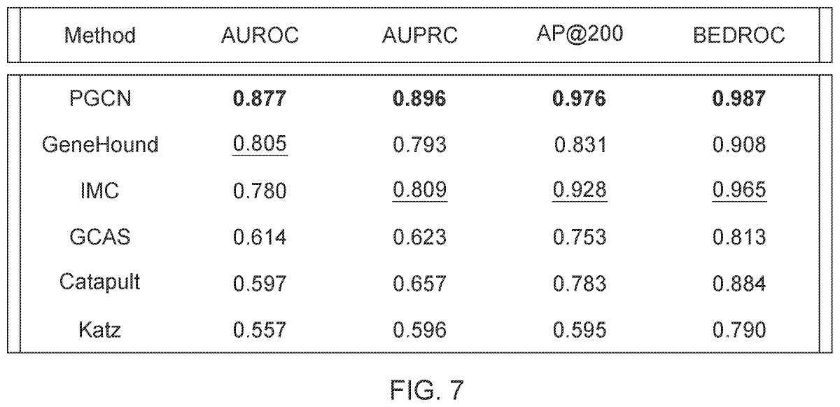

For comparing all these methods, a dataset was built from the OMIM database (Nov. 26, 2017). After preprocessing, a dataset with 12,331 genes, 3,215 diseases, and 3,988 associations was constructed. With this dataset, 10% associations were randomly hid as the testing set and the remaining 90% edges were used as the training data to evaluate the overall performance of different methods on recovering the hidden associations. The performance of the different methods discussed above is summarized in the table in FIG. 7. As shown in the table, the two matrix completion methods, GeneHound and IMC, can significantly outperform the other three network-based methods, GCAS, Catapult and Katz, across different criteria. The main reason is that they can take full advantage of the gene- and disease-specific information while the network-based methods are biased towards the network topology.

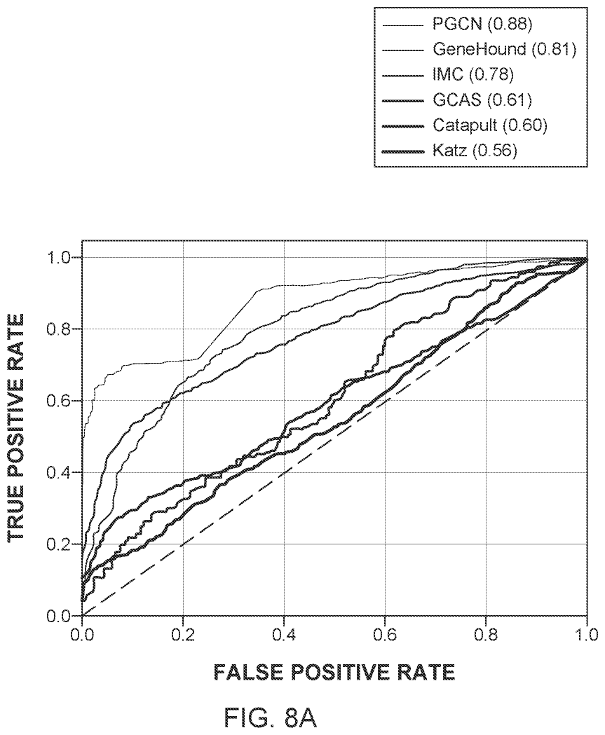

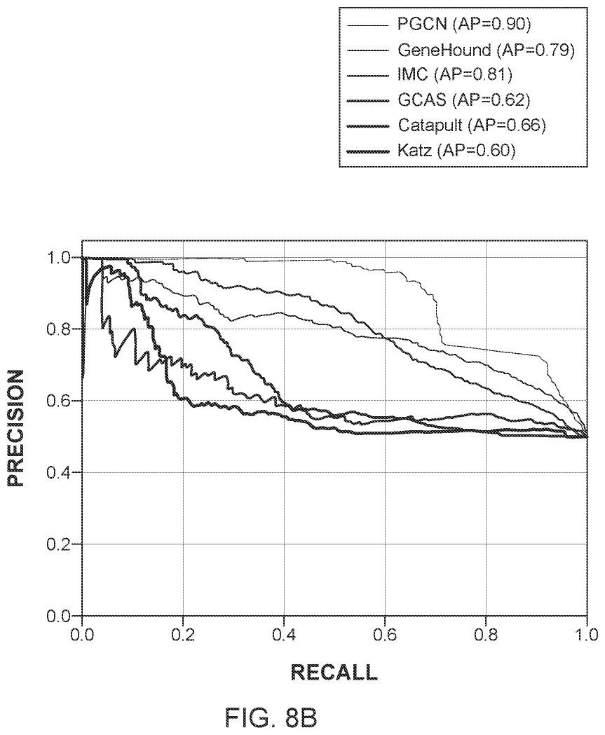

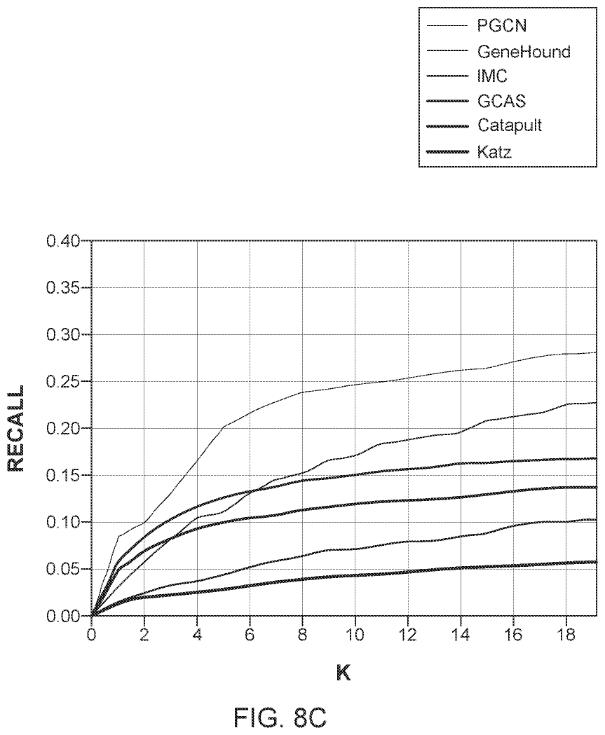

On the other hand, because the proposed method, PGCN, can utilize both the network topology information and the additional information of the nodes in a systematic and natural way, it can outperform all the state-of-the-art methods significantly and consistently across different criteria with a large margin. In terms of AUPRC, PGCN can outperform the second-best method by around 10%. The ROC curves and the PRC curves are shown in FIGS. 8A and 8B. It is clear that the PGCN method significantly outperforms all the state-of-the-art methods under all the false positive rates and all the recall values, which suggests that the PGCN method is overall a much better method.

For disease-gene prioritization, the Recall at K method is an important indicator because the top-ranked genes are candidates for further investigation. FIG. 8C shows the recall of different methods when different numbers of top predictions are considered. Interestingly, the GCAS method can perform quite well when K is very small, compared to the GeneHound, IMC, Catapult and Katz methods. However, the PGCN method is observed to be more sensitive than all the competing methods regardless of the number of top predictions to be considered. All these results demonstrate that the proposed method can outperform the other methods in recovering the hidden associations between diseases and genes.

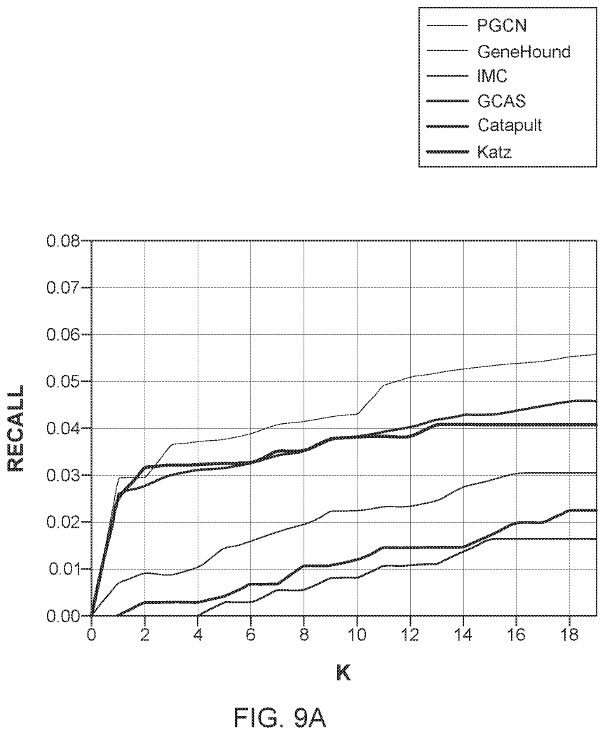

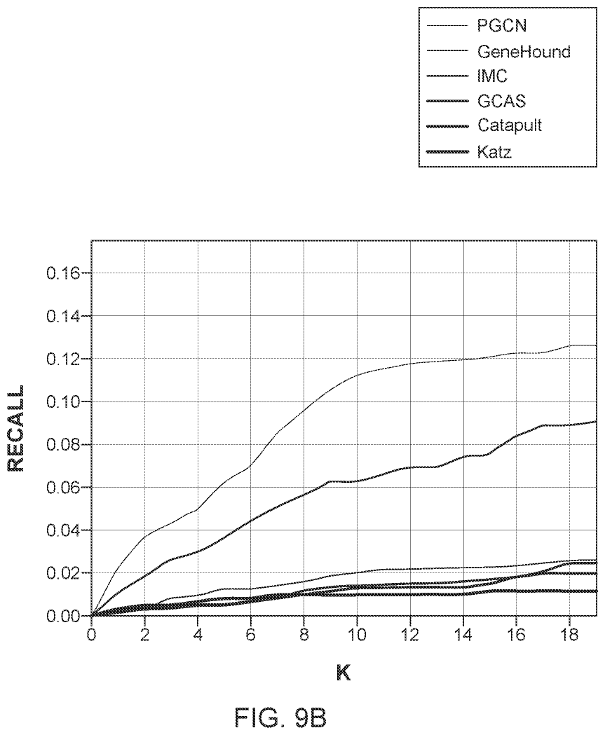

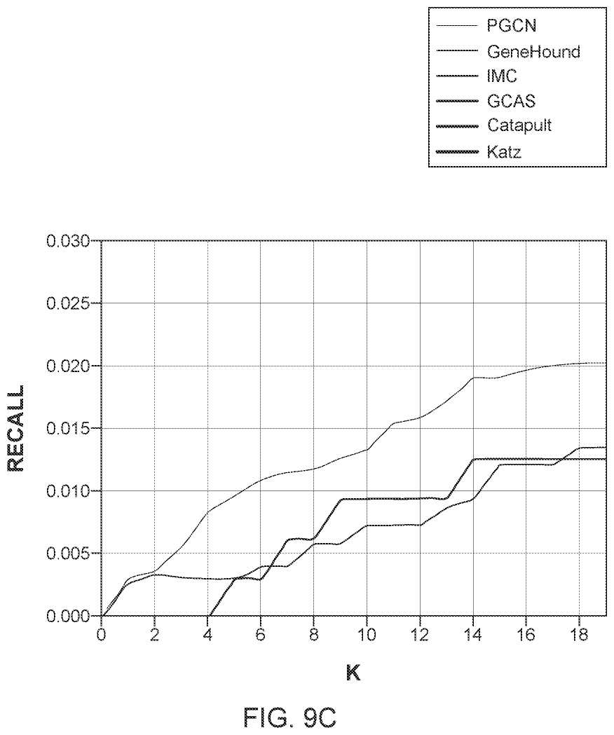

Following the idea of [8], the performance of different methods on predicting the associations of singleton genes, which are defined as those genes with only one link in the database, was checked. In the experiment performed by the inventors, the only links for the singleton genes were removed from training, which means that the methods needed to predict the associations “from scratch.” This test used the recall at K to evaluate the various methods, which is a difficult measurement because each test gene has one and only one true association. As shown in FIG. 9A, the PGCN method consistently recovers the missing associations for singleton genes, better than other methods. The inventors also noticed that the network information is important when K is small (between 1 and 10), because the improvement of the PGCN method over the network-based method is not large, which is consistent with the previous findings. However, as the number of top predictions being considered increases, the disease- and gene-specific information plays an increasingly important role, which leads to significantly better recall when K is large.

Next, the inventors evaluated the ability of the various methods to predict associations for novel diseases for which no associated genes are known. For a novel disease, all of its associations with genes were removed during training and the various methods were challenged to recover those missing associations. This task is considerably less difficult in terms of recall than recovering the associations for singleton genes because a disease can be associated with more than one gene. At the same time, this task is practically important because it is directly related to the molecular diagnosis for human diseases. As shown in FIG. 9B, the IMC method can outperform all the other previous methods with a large margin. The reason is that the IMC method is based on matrix completion techniques, which can effectively incorporate the disease-specific information. The novel method of FIG. 6, however, can not only incorporate disease- and gene-specific information, but also the known disease-gene associations in a unified framework. Furthermore, the novel method trains the disease and gene embeddings and link prediction in an end-to-end manner, and thus further significantly improves the performance over the IMC method.

To further understand how the novel method of FIG. 6 works, the inventors investigated a disease, atrioventricular septal defect-4 (AVSD4), for which its only associated gene, GATA4, was removed during the training. It was found that the PGCN method successfully recovered it with the highest score. The link between the AVSD4 and the GATA4 is built through another disease, ventricular septal defect-1 (VSD1), which is known to be associated with the GATA4. The PGCN method detected the similarity between the two diseases, AVSD4 and VSD1, according to their embeddings learned by the method, which is illustrated in FIG. 9B. However, this similarity is very difficult to be detected because in the disease similarity network, the two diseases have a wrong similarity score of 0, which suggests that they are two completely irrelevant diseases. Therefore, all the network-based methods failed to predict the association between AVSD4 and GATA4. On the contrary, the PGCN method systematically incorporates not only the network topology, but also the disease-specific information. In this particular case, the disease-specific information plays an important role in the disease embedding and thus, the PGCN method was able to detect the similarity between the two diseases in the embedding space, which led to the correct prediction on the association between AVSD4 and GATA4.

The inventors also evaluated the prediction performance of different methods for novel associations, which are defined to be the association between a disease and a gene, both of which have no association in the training set. This is the most stringent and challenging requirement. In order for a method to recover such associations, neither the disease end nor the gene end of the association can be directly used. The method must be powerful enough to effectively use the disease-and gene-specific information, and propagate the information through other diseases, genes, and their associations in the heterogeneous network. The results for this experiment are shown in FIG. 9C. As expected, the recall values of all the methods have a clear drop comparing to the two previous tasks. The inventors have found that the three network-based methods did not perform well in this task as they were unable to recall any true associations. It is suspected that the main reason for this is that the definition of novel associations makes network propagation alone extremely difficult. To support this view, the two matrix completion methods, which can take advantage of the specific information of genes and diseases, performed much better than the network-based methods. The PGCN method consistently outperforms all the competing methods, and the improvement increases with a larger K.

As a case study, the inventors have investigated the top 10 associations for breast cancer. Among these 10 genes, other than the four ground-truth breast cancer-related genes reported in the OMIM dataset, the novel model also predicted three interesting genes: Axin2, TLR4, and PTPRJ, which were reported to be related to breast cancer. For example, Axin2 was found to be included in the Wnt/β-catenin/Axin2 pathway, which can regulate the breast cancer invasion and metastasis; TLR4 was found to be overexpressed in the majority of the breast cancer samples and also related to the metastasis of breast cancer; and PTPRJ forms DEP-1/PTPRJ/CD148, which is the receptor-like protein tyrosine phosphatases (PTP), was found to be mutated or deleted in human breast cancer. These results suggest the potential application of the PGCN method on discovering new genes related to complex human diseases.

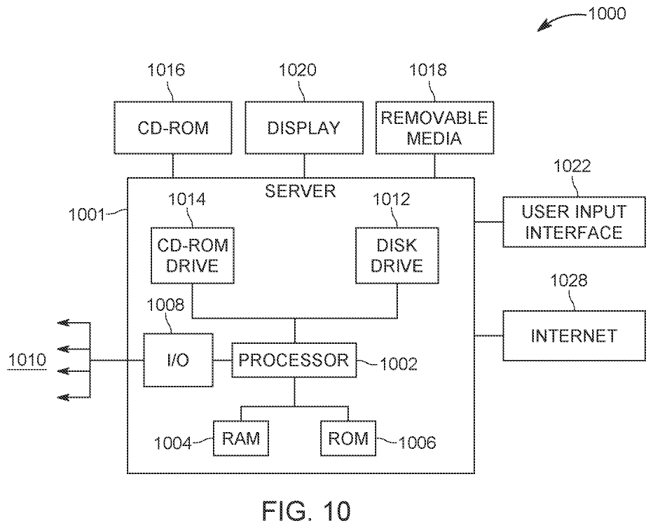

The above-discussed procedures and methods may be implemented in a computing device as illustrated in FIG. 10. Hardware, firmware, software or a combination thereof may be used to perform the various steps and operations described herein. Computing device 1000 of FIG. 10 is an exemplary computing structure that may be used in connection with such a system.

Exemplary computing device 1000 suitable for performing the activities described in the embodiments discussed above may include a server 1001. Such a server 1001 may include a central processor (CPU) 1002 coupled to a random access memory (RAM) 1004 and to a read-only memory (ROM) 1006. ROM 1006 may also be other types of storage media to store programs, such as programmable ROM (PROM), erasable PROM (EPROM), etc. Processor 1002 may communicate with other internal and external components through input/output (I/O) circuitry 1008 and bussing 1010 to provide control signals and the like. Processor 1002 carries out a variety of functions as are known in the art, as dictated by software and/or firmware instructions.

Server 1001 may also include one or more data storage devices, including hard drives 1012, CD-ROM drives 1014 and other hardware capable of reading and/or storing information, such as DVD, etc. In one embodiment, software for carrying out the above-discussed steps may be stored and distributed on a CD-ROM or DVD 1016, a USB storage device 1018 or other form of media capable of portably storing information. These storage media may be inserted into, and read by, devices such as CD-ROM drive 1014, disk drive 1012, etc. Server 1001 may be coupled to a display 1020, which may be any type of known display or presentation screen, such as LCD, plasma display, cathode ray tube (CRT), etc. A user input interface 1022 is provided, including one or more user interface mechanisms such as a mouse, keyboard, microphone, touchpad, touch screen, voice-recognition system, etc.

Server 1001 may be coupled to other devices, such as various databases, etc. The server may be part of a larger network configuration as in a global area network (GAN) such as the Internet 1028, which allows ultimate connection to various landline and/or mobile computing devices.

The disclosed embodiments provide a method for disease-gene prioritization by disease and gene embedding through graph convolutional neural networks. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] Wang, X., Gulbahce, N., and Yu, H. (2011). Network-based methods for human disease gene prediction. Brief Funct Genomics, 10(5), 280-93.
[2] Lee, I., Blom, U. M., Wang, P. I., Shim, J. E., and Marcotte, E. M. (2011). Prioritizing candidate disease genes by network-based boosting of genome-wide association data. Genome Res, 21(7), 1109-21.
[3] Guan, Y., Gorenshteyn, D., Burmeister, M., Wong, A. K., Schimenti, J. C., Handel, M. A., Bult, C. J., Hibbs, M. A., and Troyanskaya, O. G. (2012). Tissue-specific functional networks for prioritizing phenotype and disease genes. PLoS Comput Biol, 8(9), e1002694.
[4] Li, Y. and Li, J. (2012). Disease gene identification by random walk on multigraphs merging heterogeneous genomic and phenotype data. BMC Genomics, 13 Suppl 7(Suppl 7), S27.
[5] Magger, O., Waldman, Y. Y., Ruppin, E., and Sharan, R. (2012). Enhancing the prioritization of disease-causing genes through tissue specific protein interaction networks. PLoS Comput Biol, 8(9), e1002690.
[6] Kacprowski, T., Doncheva, N. T., and Albrecht, M. (2013). Networkprioritizer: a versatile tool for network-based prioritization of candidate disease genes or other molecules. Bioinformatics, 29(11), 1471-3.
[7] Nitsch, D., Tranchevent, L. C., Goncalves, J. P., Vogt, J. K., Madeira, S. C., and Moreau, Y. (2011). Pinta: a web server for network-based gene prioritization from expression data. Nucleic Acids Res, 39(Web Server issue), W334-8.
[8] Singh-Blom, U. M., Natarajan, N., Tewari, A., Woods, J. O., Dhillon, I. S., and Marcotte, E. M. (2013). Prediction and validation of gene-disease associations using methods inspired by social network analyses. PloS one, 8(5), e58977.
[9] Rao, A., Saipradeep, V., Joseph, T., Kotte, S., Sivadasan, N., and Srinivasan, R. (2018). Phenotype-driven gene prioritization for rare diseases using graph convolution on heterogeneous networks. BMC medical genomics, 11(1), 57.
[10] Zitnik, M., Agrawal, M., and Leskovec, J. (2018). Modeling polypharmacy side effects with graph convolutional networks. Bioinformatics, 34(13), i457-i466.
[11] Li, Y., Wang, S., Umarov, R., Xie, B., Fan, M., Li, L., and Gao, X. (2017). Deepre: sequence-based enzyme ec number prediction by deep learning. Bioinformatics, 34(5), 760-769.
[12] Dai, H., Umarov, R., Kuwahara, H., Li, Y., Song, L., and Gao, X. (2017). Sequence2vec: a novel embedding approach for modeling transcription factor binding affinity landscape. Bioinformatics, 33(22), 3575-3583.
[13] Kim, J.-S., Gao, X., and Rzhetsky, A. (2018). Riddle: Race and ethnicity imputation from disease history with deep learning. PLoS computational biology, 14(4), e1006106.
[14] Xia, Z., Li, Y., Zhang, B., Li, Z., Hu, Y., Chen, W., and Gao, X. (2018). DeeReCT-PolyA: a robust and generic deep learning method for PAS identification. Bioinformatics.
[15] Dai, H., Dai, B., and Song, L. (2016). Discriminative embeddings of latent variable models for structured data. arXiv.
[16] Kipf, T. N. and Welling, M. (2016). Semi-supervised classification with graph convolutional networks. arXiv.
[17] Hamilton, W. L., Ying, R., and Leskovec, J. (2017). Representation learning on graphs: Methods and applications. arXiv.

1. A method for disease-gene prioritization, the method comprising:
building a heterogenous network to include gene nodes gj and disease nodes di;
supplying additional information (x_{di}, x_{gj}) related to the gene nodes gj and the disease nodes di to generate embeddings zₖ associated with the gene nodes gj and the disease nodes di;
applying a graph convolutional neural network model G to the heterogenous network and to the embeddings zₖ to calculate aggregated embeddings zₖ₊₁; and
estimating, with an edge decoder model ED, a probability P of an edge (di, gj), between a selected gene node gj and a selected disease node di,
wherein the edge (di, gj) between the selected gene node gj and the selected disease node di is the disease-gene prioritization.

2. The method of claim 1, wherein the step of applying a graph convolutional neural network model G comprises:
aggregating, for the selected gene node, (1) embeddings z_{gk} of all gene nodes linked to the selected gene node, (2) an embedding z_{g} of the selected gene node, and (3) embeddings z_{dk} of all disease nodes linked to the selected gene node to obtain a gene feature vector h_{gk}; and
activating the gene feature vector h_{gk} with an activation function to obtain the aggregated embedding z_{g(k+1)} for the selected gene node.

3. The method of claim 2, wherein the step of applying a graph convolutional neural network model G further comprises:
aggregating, for the selected disease node, (1) embeddings z_{dk} of all disease nodes linked to the selected disease node, (2) an embedding z_{d} of the selected disease node, and (3) embeddings z_{gk} of all gene nodes linked to the selected disease node to obtain a disease feature vector h_{dk}; and
activating the disease feature vector h_{dk} with the activation function to obtain the aggregated embedding z_{d(k+1)} for the selected disease node.

4. The method of claim 3, wherein the step of aggregating, for a selected gene node or for a selected disease node, uses a different weight for each type of embedding.

5. The method of claim 4, further comprising:
training the graph convolutional neural network model G and the edge decoder model ED for each of the different weight.

6. The method of claim 3, wherein the step of estimating comprises:
calculating the probability P as a sigmoid function applied to a product of (1) the aggregated embedding of the selected gene node, (2) a weight of the edge decoder model, and (3) the aggregated embedding of the selected disease node.

7. The method of claim 6, further comprising:
applying a cross-entropy loss function L to the edge decoder model ED to calculate a final probability P_{f} of the edge (di, gj).

8. The method of claim 1, wherein the additional information includes one or more of an Online Mendelian Inheritance in Man, disease ontology, associations in other species, human mRNA co-expressions, protein-protein interactions, protein complex, comparative genomics interaction, and disease similarity network.

9. The method of claim 1, wherein the heterogenous network includes a gene network, a disease network, and a gene-disease network.

10. The method of claim 1, wherein the step of building comprises:
linking each gene node gj to other known gene nodes;
linking each disease node di to other known disease nodes; and
linking each gene node gj to the disease node di if such a link is known.

11. The method of claim 1, further comprising:
initializing the embeddings with the additional information.

12. A computing device for producing a disease-gene prioritization, the device comprising:
an input/output interface for receiving additional information (x_{di}, x_{gj}) related to gene nodes gj and disease nodes di to generate embeddings zₖ associated with the gene nodes gj and the disease nodes di; and
a processor connected to the input/output interface and configured to,
build a heterogenous network made by the gene nodes gj and the disease nodes di;
apply a graph convolutional neural network model G to the heterogenous network and the embeddings zₖ to calculate aggregated embeddings zₖ₊₁; and
estimate, with an edge decoder model ED, a probability P of an edge (di, gj), between a selected gene node gj and a selected disease node di,
wherein the edge (di, gj) between the selected gene node gj and the selected disease node di is the disease-gene prioritization.

13. The device of claim 12, wherein the processor is further configured to:
aggregate, for the selected gene node, (1) embeddings z_{gk} of all gene nodes linked to the selected gene node, (2) an embedding z_{g} of the selected gene node, and (3) embeddings z_{dk} of all disease nodes linked to the selected gene node to obtain a gene feature vector h_{gk}; and
activating the gene feature vector h_{gk} with an activation function to obtain the aggregated embedding z_{g(k+1)} for the selected gene node.

14. The device of claim 13, wherein the step of applying a graph convolutional neural network model G further comprises:
aggregating, for the selected disease node, (1) embeddings z_{dk} of all disease nodes linked to the selected disease node, (2) an embedding z_{d} of the selected disease node, and (3) embeddings z_{gk} of all gene nodes linked to the selected disease node to obtain a disease feature vector h_{dk}; and
activating the disease feature vector h_{dk} with an activation function to obtain the aggregated embedding z_{d(k+1)} for the selected disease node.

15. The device of claim 14, wherein the step of aggregating, for the selected gene node or for the selected disease node, uses a different weight for each type of embedding.

16. The device of claim 15, wherein the processor is further configured to:
train the graph convolutional neural network model G and the edge decoder model ED for each of the different weights.

17. The device of claim 14, wherein the processor is further configured to:
calculate the probability P as a sigmoid function applied to a product of (1) the aggregated embedding of the selected gene node, (2) a weight of the edge decoder model, and (3) the aggregated embedding of the selected disease node.

18. The device of claim 17, wherein the processor is further configured to:
apply a cross-entropy loss function L to the edge decoder model ED to calculate a final probability P_{f} of the edge (di, gj).

19. The device of claim 12, wherein the processor is further configured to:
link each gene node gj to other known gene nodes;
link each disease node di to other known disease nodes; and
link each gene node gj to the disease node di if such a link is known.

20. A method for training a graph convolutional neural network model G for disease-gene prioritization, the method comprising:
building a heterogenous network from gene nodes gj and disease nodes di;
supplying additional information (x_{di}, x_{gj}) related to the gene nodes gj and the disease nodes di to generate embeddings zₖ associated with the gene nodes gj and the disease nodes di;
applying the graph convolutional neural network model G to the heterogenous network and the embeddings zₖ to calculate aggregated embeddings zₖ₊₁;
estimating, with an edge decoder model ED, a probability P of an edge (di, gj), between a selected gene node gj and a selected disease node di; and
repeating the above steps until the probability P is one for a known connection between the selected gene node gj and the selected disease node di,
wherein the edge (di, gj) between the selected gene node gj and the selected disease node di is the disease-gene prioritization.
